(19) United States
(12) Patent Application Publication
(10) Pub. No.: US 2020/0398001 A1
(43) Pub. Date: Dec. 24, 2020

(54) PEN NEEDLE AND INNER SHIELD

(71) Applicant: Becton, Dickinson and Company, Franklin Lakes, NJ (US)

(72) Inventor: Peter GIRGIS, East Brunswick, NJ (US)

(73) Assignee: Becton, Dickinson and Company, Franklin Lakes, NJ (US)

(21) Appl. No.: 16/968,790

(22) PCT Filed: Feb. 11, 2019
(86) PCT No.: PCT/US19/17423

§ 371 Date: Aug. 10, 2020

Related U.S. Application Data
(60) Provisional application No. 62/629,522, filed on Feb. 12, 2018.

(51) Int. Cl. A61M 5/32

(52) U.S. Cl. CPC A61M 5/3204; A61M 2205/586; A61M 5/3293

(57) ABSTRACT

A pen needle assembly (30) includes pen needle hub (32) and an inner shield (34) for covering a needle (46) extending from the needle hub (32). The needle hub (32) and inner shield (34) include a coupling mechanism (52, 54) to assist in removing the inner shield from the needle hub. In one embodiment, the needle hub (32) includes a threaded connection (52) for mating with a threaded connection (54) on the inner shield (34) to assist in removing the inner shield by a twisting motion to disconnect the inner shield from the needle hub. The threaded connection (52, 54) can have a pitch greater than an axial length of a post on the needle hub.

This application claims the benefit of U.S. provisional patent application No. 62/629,522, filed on Feb. 12, 2018, which is hereby incorporated by reference in its entirety.

### FIELD OF THE INVENTION

The present invention relates to a pen needle assembly for use with a delivery device, such as a pen delivery device. The pen needle assembly includes an inner shield having an attachment mechanism where the inner shield can be removed and replaced with reduced effort compared to conventional pen needles. The attachment mechanism can be luer lock type coupling.

### BACKGROUND OF THE INVENTION

Insulin and other injectable medications are commonly delivered with drug delivery pens, whereby a disposable pen needle hub is attached to the pen to access the drug container and allow fluid egress from the container through the needle into the patient.

Various pen needle delivery devices are known in the art for dispensing the substance to the patient. The delivery devices often use a disposable needle hub having a cannula or needle extending from a patient end of the hub for inserting into the patient. A non-patient end of the hub is coupled to the pen delivery device for delivering the substance to the patient.

The pen needle is often packaged in a container containing several loose needle hubs. A needle hub is selected from the package and attached to the pen needle delivery device for injecting the patient and then removed to be discarded. The needle hub package includes an outer cover that encloses the needle hub and a removable seal on the open end that is peeled from the outer cover to open the cavity so that the needle hub can be removed. The needle hub can have threaded non-patient end that is threaded onto the delivery device. The delivery device with the attached needle hub is then removed from the outer cover. An inner needle shield is attached to the needle hub to cover the needle until the device is ready for use. The shield is removed to expose the needle for use to deliver the substance to the patient. After use, the needle hub can be inserted back into the outer cover to enclose the exposed needle. The pen delivery device is separated from the needle hub leaving the needle hub within the outer cover.

The prior devices require the use of both hands to connect to and remove the needle hub from the delivery device. During the placement back into the outer cover, the cannula is exposed and provides an increased risk of accidental needle stick. The manual operation of holding the outer cover while positioning the spent needle hub and cannula into the cavity of the outer cover can be difficult without accidental needle stick.

Existing pen needle assemblies are disclosed in U.S. Patent Application Publication Nos. 2006/0229562 to Marsh et al. and 2007/0149924 to R. Marsh, the entire contents of both of which are hereby incorporated by reference.

Although the prior devices have been suitable for the intended use, there is a continuing need in the industry for improved packaging for a pen needle hub assembly.

### SUMMARY OF THE INVENTION

The present invention is directed to a pen needle assembly for use with an injection device, where the pen needle has an inner shield that can be removed to expose the needle. The invention is also directed to a method of use of the pen needle assembly and attaching the pen needle to the injection device. The inner shield can be connected to the needle hub a luer lock type connector mechanism.

The pen needle assembly includes a needle hub and an inner shield that is coupled to the needle hub to cover the needle mounted to the needle hub. A mechanical coupling is provided between the inner shield and the needle hub so that inner shield can be easily separated from the needle hub. The mechanical coupling can be configured to assist the user in separating the inner shield from the needle hub with limited effort by the user.

In one embodiment, the inner shield can be mechanically connected to the needle hub by a threaded coupling whereby the inner shield is separated from the needle hub by a twisting motion. One example is a threaded coupling configured so that the inner shield is separated from the needle hub by rotating the inner shield less than one full revolution of the inner shield relative to the needle hub. A small rotation of the inner shield relative the needle hub enables the user to quickly and easily remove the inner shield from the needle hub without pulling the inner shield axially from the needle hub.

The features of the invention are basically attained by providing a pen needle assembly including a needle hub, inner shield, and a coupling mechanism between the inner shield and the needle hub. The needle hub has a needle post supporting a needle, where the needle post has an outer surface and a distal end, and where the needle extends from the distal end of the needle post. The inner shield is configured for coupling to an outer surface of the needle post and covering the needle during storage and packaging of the pen needle. The inner shield can have an outer surface with at least one textured gripping surface to assist the user in rotating the needle shield with respect to the needle hub. The coupling mechanism is a mechanical coupling mechanism for connecting the inner shield to the post where the inner shield can be separated and removed from the post.

The features are further provided by a pen needle assembly comprising a needle hub, inner shield and outer cover. The needle hub includes a needle post extending from the hub for supporting a needle or cannula. The needle post has an outer surface and a distal end, where the needle extends from the distal end of the needle post. The inner shield has a threaded coupling mechanism for coupling to an outer surface of the needle post and covering the needle. The inner shield has an outer surface with at least one textured gripping surface. The outer cover is coupled to an outer surface of the needle hub covering the needle hub and inner shield. The outer cover and inner shield are removable from the needle hub.

The features of the apparatus are also attained by providing a method of assembling a pen needle assembly on a pen needle delivery device, where the pen needle assembly includes a needle hub with a post supporting a needle, an inner shield threaded to the post, and an outer cover covering the needle hub and inner shield. The method comprises coupling the pen needle hub to the pen needle The outer cover is removed to expose the inner shield, and the inner shield is rotated relative to the post to disconnect the inner shield from the post. The inner shield is removed to expose the needle.

After use of the needle hub and needle, the needle hub while attached to the delivery pen is inserted into the outer cover where the needle hub can be gripped by a friction fit or interference fit to the outer cover so that the pen needle delivery device can be separated from the needle hub without the operator handling the needle hub thereby reducing the risk of inadvertent needle stick.

The objects, advantages, and salient features of the invention will become apparent from the following detailed description, which, taken in conjunction with the annexed drawings, discloses exemplary embodiments of the invention.

### BRIEF DESCRIPTION OF THE DRAWINGS

The above benefits and other advantages of the various embodiments of the present invention will be more apparent from the following detailed description of exemplary embodiments of the present invention and from the accompanying figures, in which:

FIG. 1 is an exploded perspective view of a pen needle delivery device in one embodiment showing the pen needle assembly that includes a needle hub supporting a needle, an inner shield, and an outer cover;

FIG. 2 is a cross-sectional view of a pen needle assembly;

FIG. 3 is an exploded view showing the delivery device and the pen needle in an embodiment that includes a needle hub and an inner shield;

FIG. 4 is a front elevational view of the needle hub in one embodiment;

FIG. 5 is an elevational view of the inner shield of the pen needle assembly;

FIG. 6 is a side view of the needle hub showing the internal threads in the hub and showing the threads on the post in phantom lines;

FIG. 7 is a perspective view of the inner shield showing the internal threads in phantom lines;

FIG. 8 is a top view of the inner shield; and

FIG. 9 is a bottom view of the inner shield.

Throughout the drawings, like reference numbers will be understood to refer to like parts, components, and structures.

### DETAILED DESCRIPTION OF THE EXEMPLARY EMBODIMENTS

The present invention is directed to a pen needle assembly apparatus for use with an injection delivery device such a pen needle delivery device. The invention is further directed to a pen needle assembly having an inner shield that is configured for assisting in the removal and replacement of the inner shield on the needle hub.

Reference to embodiments of the present invention are illustrated in the accompanying drawings, wherein like reference numerals refer to like elements throughout. The embodiments described herein exemplify, but do not limit, the present invention by referring to the drawings. The exemplary embodiments are presented in separate descriptions, although the individual features and construction of these embodiments can be combined in any number of ways to meet the therapeutic needs of the user.

It will be understood by one skilled in the art that this disclosure is not limited in its application to the details of construction and the arrangement of components set forth in the following description or illustrated in the drawings. The embodiments herein are capable of being modified, practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. Further, terms such as up, down, bottom, and top are relative, and are employed to aid illustration, but are not limiting. The features or elements of the different embodiments can be combined with one another in a manner that is not inconstant with the embodiment to obtain a different operative embodiment.

The needle hub assembly enables the convenient and easy assembly and removal of the needle hub on the pen delivery device. The inner shield of the needle hub is easily removed with reduced effort for patients with reduced strength or dexterity such a patients exhibiting neuropathy in the hands or fingers. In one embodiment, the inner shield is separated from the needle hub by a twisting motion of the inner shield relative to the needle hub to release the inner shield. The inner shield of the prior devices have a length of about 200 mm and a diameter of about 3 mm that is pulled from the needle axially from the needle hub. The small dimensions of the inner shield can be difficult to grip for some patients.

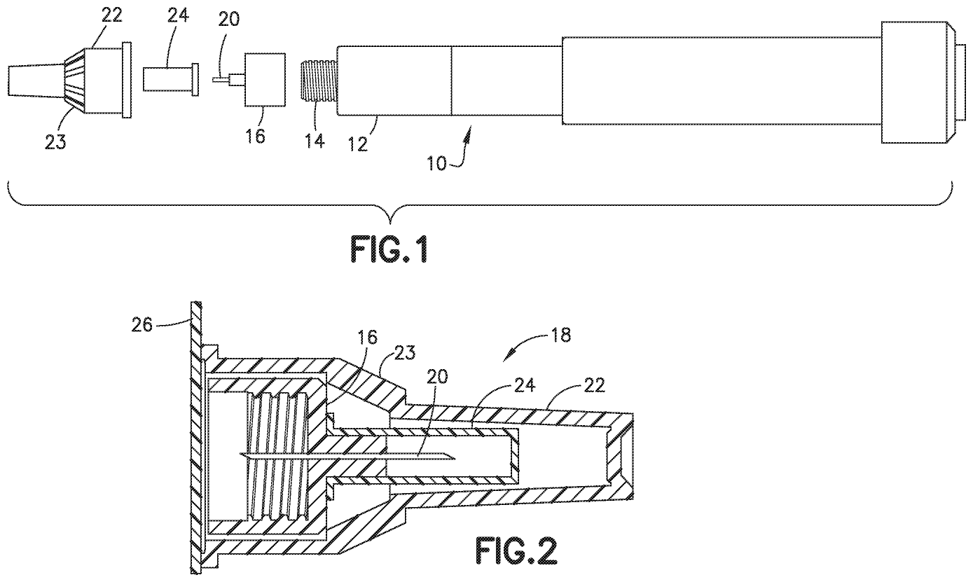

Delivery device 10, as shown in FIG. 1 typically comprises a dose knob/button, an outer sleeve 12, a threaded end 14, and a cap. The dose knob/button allows a user to set the dosage of medication to be injected. The outer sleeve 12 is gripped by the user when injecting medication. The cap is used by the user to securely hold the pen injector device 10 in a shirt pocket or other suitable location and provide cover/protection from accidental needle injury.

In standard pen needle delivery devices the dosing and delivery mechanisms are all found within the outer sleeve 12 and are not described in greater detail here as they are understood by those knowledgeable of the prior art. A medicament cartridge is typically attached to a standard pen injector housing by known attachment means. The distal movement of a plunger or stopper within the medicament cartridge causes medication to be forced into the reservoir housing. The medicament cartridge is sealed by a septum and punctured by a septum penetrating needle cannula located within a reservoir or housing. Reservoir housing is preferably screwed onto the medicament cartridge although other attachment means can be used. The pen needle delivery device can be a standard pen delivery device known in the industry so that the pen needle delivery device is not shown in detail.

The pen needle assembly 18 as shown in FIG. 2 includes a needle hub 16 supporting a cannula 20, an outer cover 22, and an inner shield 24. A protective seal 26 is attached to the open end of the outer cover as shown in FIG. 2 to enclose the needle hub and cannula to maintain a clean and sterile condition. The seal 26 can be a label or other closure member that can be easily peeled from the outer cover to access the needle hub during use.

The delivery device 10 is connected to needle hub 16 shown in FIG. 1 that has a connecting non-patient end with internal threads that screw onto the threaded end 14 of the delivery device 10. The needle 20 extends from the patient end of the needle hub 16 for delivering the substance to the patient. The outer cover 22 covers the needle to protect the patient from accidental needle stick before and after use. The outer cover 22 includes ribs 23 to assist in gripping the outer cover during use. The inner shield 24 is provided over a post extending from the end of the needle hub 16 to enclose the needle. During use, the needle hub 16 is connected to the pen delivery device and the inner shield is removed. After use, the outer cover is generally placed back on needle hub to cover the needle. The needle hub with the cover is then removed from the pen needle delivery device and discarded.

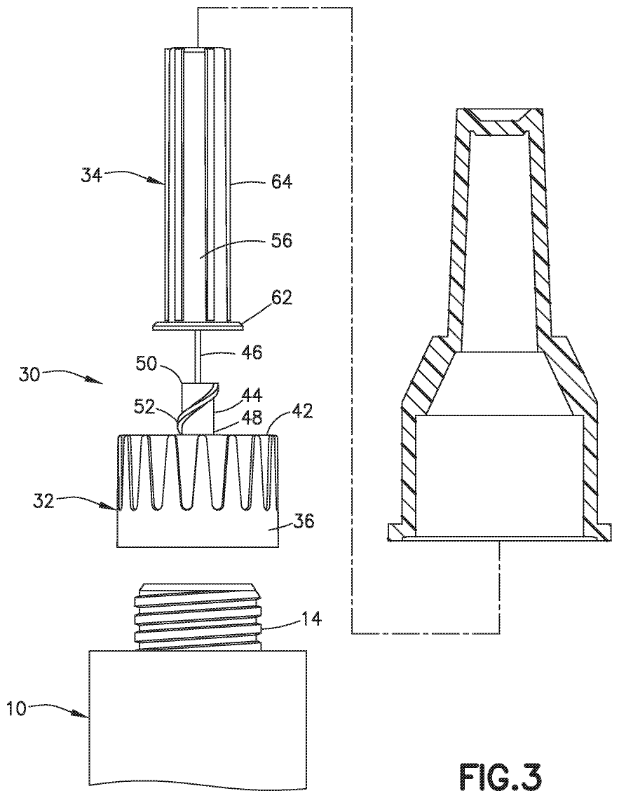

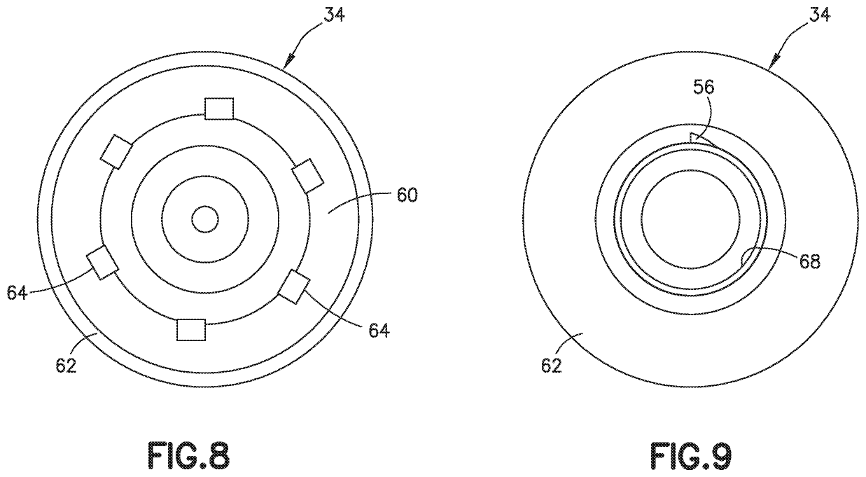

Referring to FIGS. 3-9, the pen needle assembly 30 in one embodiment of the invention is configured for coupling to the pen needle delivery device 10 as shown in FIG. 1. The pen needle assembly 30 as shown in FIG. 3 includes a needle hub 32 and an inner shield 34. An outer cover is coupled to the needle hub 32 in the same manner as shown in FIGS. 1 and 2.

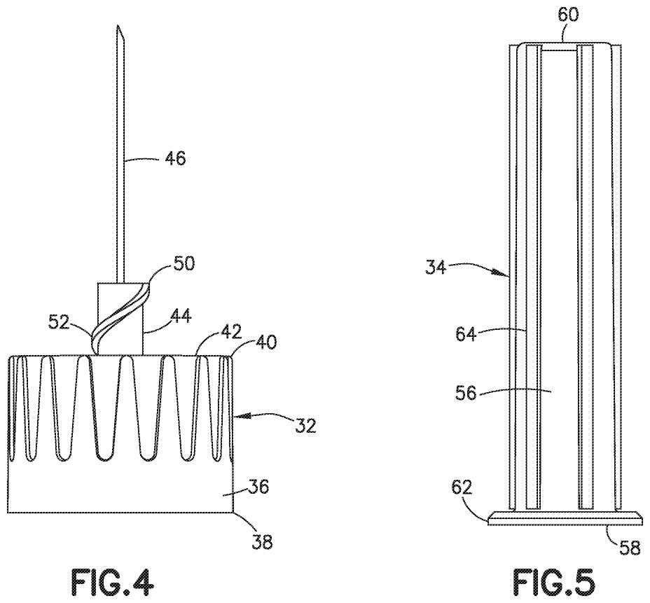

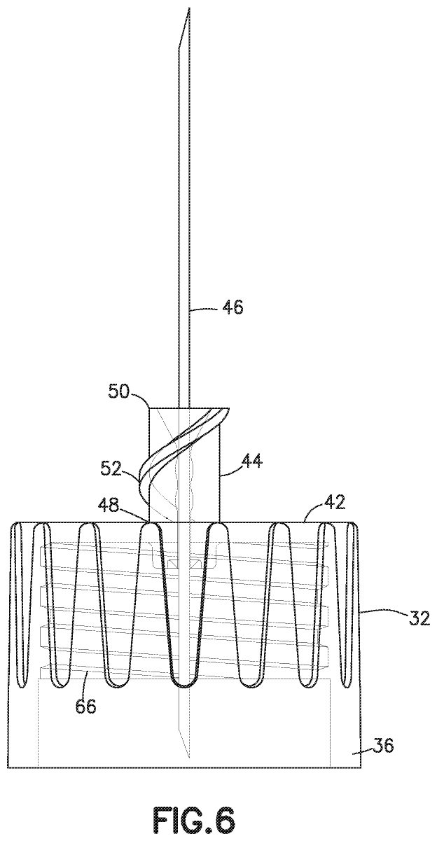

As shown in FIG. 3 and FIG. 4, the needle hub 32 has an outer side wall 36 with an open proximal end 38 for coupling with the delivery device 10 and a closed distal end 40 defining a patient contact end. Internal threads 66 shown in phantom lines in FIG. 6 are provided for coupling the needle hub to the pen needle delivery device as known in the art. The closed distal end 40 of the needle hub 32 is formed by a top wall 42. In the embodiment shown, the top wall 42 has a substantially flat configuration extending in a plane perpendicular to a longitudinal axis of the needle hub 32. In other embodiments, the top wall 42 can have a contoured or convex shape.

A centrally located post 44 extends from the needle hub 32 for supporting a needle 46 or cannula. The post 44 extends in the axial direction with respect to the axis of the needle hub 32 and projects from the top wall 42. The post has a proximal end 48 connected to the top wall 42 and a distal end 50. The needle 46 projects from the distal end 50 of the post 50. As shown in FIG. 6, the needle 46 extends through an axial passage in the post 44 and the top wall 42 and projects into the inner space of the needle hub 32 for connecting to the delivery device. The post 44 can have a longitudinal dimension of about 5 to 10 mm and typically about 5-7 mm. The diameter of the post 44 provides sufficient strength to prevent bending during use and to support the needle 46. Typically the post has a diameter of about 3-4 mm. The length of the post 44 can be modified as needed depending on the intended use, the desired depth of penetration, and the delivery device.

The needle 46 has a suitable length projecting from the distal end of the post to attain the desired depth of penetration. The needle 46 can have an exposed length of about 4.0-12.7 mm and typically about 6.0-8.0 mm extending from the distal end of the post. In one embodiment, the needle can have an effective length of about 3-4 mm. The needle length is selected according to the intended depth of penetration. The needle is typically 28-32 gauge although other gauges can be used.

The inner shield 34 includes a coupling mechanism that cooperates with the needle hub 32 to connect the inner shield 34 to the needle hub 32 in a manner that is easily separated. In the embodiment shown, the post 44 includes a coupling mechanism that cooperates with the inner shield 34 to connect the inner shield securely to the post and needle hub during storage while enabling quick separation from the post and needle hub when ready for use. The inner shield 34 is also easily attached back on the needle hub after use to reduce the occurrence of accidental needle stick.

The coupling mechanism in one embodiment is a threaded coupling between the inner shield 34 and the post 44 that is able secure the inner shield 34 to the post 44 during storage and prior to use. The threaded coupling enables the user to remove the inner shield quickly and easily to expose the needle 46 for injecting the substance to the patient. During use, the pen needle assembly is coupled to the pen needle delivery device and the outer cover is removed to expose the inner shield 34. When the assembly is ready for use, the inner shield 34 is removed to expose the needle. The inner shield 34 is typically quite small relative to the fingers of the patient so that coupling mechanism enables the quick and easy removal of the inner shield without the inner shield slipping between the hands or fingers of the user. The inner shield 34 has a length of about 12-18 mm and typically about 15 mm with an outer diameter of about 2-5 mm.

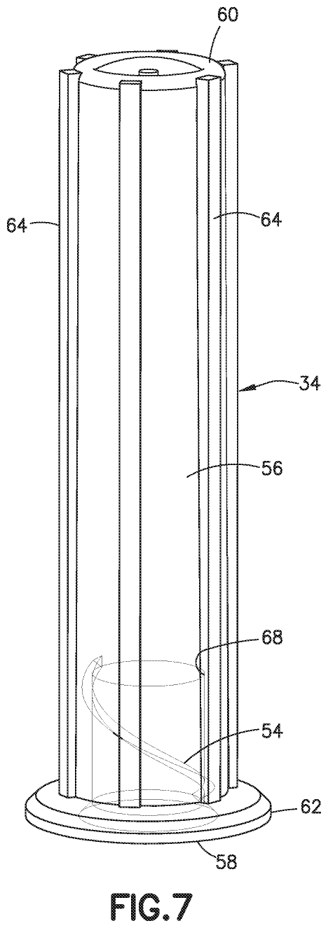

In one embodiment, the coupling mechanism includes an external thread 52 on the outer surface of the post 44. The inner shield 34 as shown in FIG. 7 includes an inner thread 54 complementing the external thread 52 on the post 44. Referring to FIGS. 3 and 4, the external thread 52 extends from the top wall 42 at the proximal end 38 of the post 44 to the distal end 40 of the post 44. In the embodiment shown, a single thread is provided although the more than a single thread can be used. The external thread 52 and internal thread 54 are configured to enable quick separation the inner shield 34 from the post 44 with minimal effort and minimal rotation of the inner shield relative to the post. The shape and cross section of the thread 52 can be square, triangular or other shape and configuration. The internal thread 54 of the inner shield 34 typically has a shape and configuration complementing the shape and configuration of the thread 52.

Thread 52 generally terminates at or close to the distal end 40 of the post 44 shown in FIG. 4. In one embodiment the bottom end of the thread 52 can terminate at a point spaced from the proximal end 48 of the post 44 as long as the thread 52 has a length and dimension sufficient to mate with the inner surface of the inner shield, provide sufficient retaining of the inner shield, and allow quick and easy separation of the inner shield from the post.

In one embodiment, the thread 52 has a lead and pitch not less than the axial length of the post 44. As shown in the drawings, the thread 52 has lead and pitch greater than the axial length of the post 44 so that the pitch of the thread 52 is greater than one revolution around the circumference of the post 44 and the thread 52 has a length circling the post 44 less than one full revolution. The thread 52 has a pitch at least equal to the axial length of the post 34 and typically greater than the axial length of the post 44. In the embodiment shown, the thread 52 has a pitch whereby the thread 52 extends about ⅔ to about ¾ around the circumference of the post so that the thread makes less than one full revolution around the post. In one embodiment the post 44 has an axial length of about 5 mm where the thread 52 has a pitch of about 5-8 mm and generally about 6-8 mm. In other embodiments, the thread 52 has a pitch of about 5-10 mm when the post has a length of about 5 mm. The ratio of the pitch of the thread to the length of the post can be about 1:1 to 1:6.

In the embodiment shown, the thread 52 has a pitch greater than the axial length or height of the post so that the thread extends around the circumference less than one revolution and generally about ¾ of a revolution with respect to a circumference of the post. The thread 52 can have a pitch of about 1 to 2 times the axial longitudinal dimension of the post 44 and generally about 1 to 1.5 times the longitudinal dimension of the post. In an alternative embodiment, the thread 52 has a pitch of about 1.2 to 1.3 times the longitudinal dimension of the post 44. The pitch is selected to allow quick and easy separation and attachment to the needle hub while maintaining a secure attachment to the post 44.

The inner shield 34 includes a side wall 56 with an inner surface 68, an open proximal end 58, and a closed distal end 60 as shown in FIGS. 5 and 7-9. A flange 62 extends radially outward from the side wall 56 at the proximal end 58 to assist in handling the inner shield 34 and to contact the top wall 42 of the needle hub 32 when the inner shield is positioned on the needle hub 32.

The thread 54 of the inner shield 34 is formed on an inner surface of the side wall 56 and extends from the proximal end 58 toward the distal end 60 as shown in FIG. 7. The thread 54 is in the form of a spiral groove with a pitch corresponding substantially to the pitch of the thread 52. The length of the thread 54 is at least sufficient to mate with the thread 52 when coupled so the inner shield 34 is securely attached to the post 44 and the proximal end of the inner shield 34 mates with the top wall 42 of the needle hub 32. The axial length of the thread 52 is at least equal to the axial length of the post 44. In one embodiment, the thread 54 has a length to extend about ⅔ to about ¾ around the inner surface of the inner shield 34. In one embodiment, the thread 54 extends less than one complete revolution around the inner surface of the inner shield 34. The thread 54 can have a pitch of about 5-8 mm and typically about 6-8 mm to complement the pitch of the thread 52 on the post 44.

The side wall 56 of the inner shield 34 in one embodiment has a substantially cylindrical shape with a cylindrical outer surface. Gripping members are provided on the outer surface of the side wall 56 to assist in gripping the inner shield 34 and rotating the inner shield 34 relative to the post 44. In the embodiment shown, the gripping members are formed by a plurality of longitudinally extending ribs 64 that project radially outward from the side wall 56 a distance to enable firmly gripping of the inner shield by the user. In one embodiment, the ribs 64 have a radial dimension with a ratio of about 1:3 to 1:6 relative to the radius of the inner shield. In another embodiment, the ribs 64 have a radial dimension of about ¼ to ⅕ of the radius of the inner shield. In other embodiments, gripping member can have other shapes or configurations. The ribs 64 can have a longitudinal length suitable for gripping by the user. In the embodiment shown, the ribs 64 have a longitudinal length extending between the distal end 60 and the proximal end 58 at the flange 62. As shown, the inner shield 34 has a length to cover the post and needle without interfering with the needle.

The threaded coupling mechanism allows the inner shield to be removed by a short twist or small rotation of the inner shield so that the inner shield is easily released and pulled free from the post. Some patients that exhibit neuropathy in the hands or fingers can have difficulty in pulling the inner shield due the small dimensions of the inner shield and the tight friction fit on the post. Conventional pen needle assemblies have a smooth outer surface and are attached to the post of the needle hub by friction fit that can be difficult to grip and pull free from the post. The threaded coupling also allows replacement on the post to cover the needle after use by a secure attachment to reduce the occurrence of accidental needle stick.

The ribs 64 on the outer surface of the inner shield 34 enable the patient to grip the inner shield so that the inner shield can be rotated to disconnect the inner shield from the needle hub 32. The threaded coupling between the inner shield 34 and the post 44 has a pitch so that the inner shield is separated from the post 44 by a twist of less than one full rotation of the inner shield relative to the needle hub 32. The thread 52 on the post 44 extends to the distal end 50 so that when the thread 54 on the inner shield 34 reaches the end of the post, the inner shield disengages from the post 44. In one embodiment, the inner diameter of the inner shield 34 complements the outer diameter and surface of the post 44 so that inner shield can be retained on the post without requiring excessive force to rotate the inner shield. In one embodiment, the clearance between the post 44 and the inner shield 34 is sufficient to minimize friction between the outer surface of the post and the inner surface of the inner shield so that gripping between the inner shield and the post are provided primarily by the threaded connection.

In one embodiment the inner diameter of the cavity of the inner shield is slightly less than an outer diameter of the post to limit resistance in rotating the inner shield relative to the needle hub. The threads 52 and 54 are configured to provide sufficient frictional engagement to retain the inner shield on the post without interfering with the separation of the inner shield.

In the embodiment shown, the coupling mechanism has complementing threads although other coupling mechanisms can be used. In another embodiment, the post can include a detent projecting outward from the side wall to mate with a spiral groove formed on the inner surface of the inner shield. In other embodiments, the inner surface of the inner shield can have a detent or spiral thread projecting radially inward toward the center axis of the inner shield to mate with a spiral groove formed in the outer surface of the post. In still further embodiments, the coupling mechanism can be a bayonet type connection where the post or the inner shield include an L-shaped groove that mates with detent on the other of the post or inner shield.

The method of use of the pen needle assembly attaches the pen needle assembly to the delivery pen for injecting the substance to the patient. The outer cover is removed from the needle hub to expose the inner shield. The patient is then able to rotate or twist the inner shield with respect to the needle hub and separate the inner shield from the hub to expose the needle. After use, the outer cover can be replaced on the needle hub for removing the used needle hub from the pen needle delivery device and discarding the used needle hub.

The foregoing embodiments and advantages are merely exemplary and are not to be construed as limiting the scope of the present invention. The description of an exemplary embodiment of the present invention is intended to be illustrative, and not to limit the scope of the present invention. Various modifications, alternatives, and variations will be apparent to those of ordinary skill in the art, and are intended to fall within the scope of the invention. It is particularly noted that the features of different embodiments and claims may be combined with each other as long as they do not contradict each other. Accordingly, all such modifications are intended to be included within the scope of this invention as defined in the appended claims and their equivalents.

1. A pen needle assembly comprising:
a needle hub having a needle post supporting a needle, said needle post having an outer surface and a distal end, said needle extending from said distal end of said needle post;
an inner shield for coupling to an outer surface of said needle post and covering the needle, said outer shield having an outer surface with at least one textured gripping surface; and
a mechanical coupling mechanism for connecting said inner shield to said post.

2. The pen needle assembly of claim 1, wherein said inner shield includes a plurality of ribs defining a textured gripping surface.

3. The pen needle assembly of claim 2, wherein said plurality of ribs extend in a longitudinal direction with respect to a longitudinal dimension of said inner shield.

4. The pen needle assembly of claim 2, wherein said inner shield has a side wall with an open proximal end and a closed distal end, and where said closed proximal end includes a flange extending radially outward from said side wall.

5. The pen needle assembly of claim 4, wherein said ribs extend between said distal end of said side wall and said flange.

6. The pen needle assembly of claim 4, wherein said coupling mechanism on said outer shield is a threaded coupling.

7. The pen needle assembly of claim 4 wherein said outer surface of said post includes a spiral thread, and said inner shield has an inner surface with a spiral thread complementing said spiral thread on said post.

8. The pen needle assembly of claim 7, wherein said spiral thread on said post extends between said flange and said distal end of said post, and said spiral thread has a pitch greater than or equal to a longitudinal length of said post.

9. The pen needle assembly of claim 7, wherein said spiral thread on said post has a pitch greater than a longitudinal length of said post.

10. The pen needle assembly of claim 7, wherein said spiral thread on said post has a length extending about ½ to ⅔ of a circumference of said post.

11. The pen needle assembly of claim 7, wherein said spiral thread on said post has a pitch of about 1.0 to 1.5 times the longitudinal length of said post.

12. A pen needle assembly comprising:
a needle hub having a needle post extending from said hub and supporting a needle, said needle post having an outer surface and a distal end, said needle extending from said distal end of said needle post;
an inner shield having a threaded coupling mechanism for coupling to an outer surface of said needle post and covering the needle, said inner shield having an outer surface with at least one textured gripping surface; and
an outer shield coupled to an outer surface of said needle hub covering said needle hub and inner shield, wherein said outer shield and inner shield are removable from said needle hub.

13. The pen needle assembly of claim 12 wherein said outer surface of said post includes a spiral thread, and said threaded coupling mechanism of said inner shield is a spiral thread on an inner surface of said inner shield complementing said spiral thread on said post.

14. The pen needle assembly of claim 13, wherein said spiral thread on said post extends between a proximal end of said post and said distal end of said post, and where said spiral thread has a pitch greater than or equal to a longitudinal length of said post.

15. The pen needle assembly of claim 13, wherein said spiral thread on said post has a pitch greater than a longitudinal length of said post.

16. The pen needle assembly of claim 13, wherein said spiral thread on said post has a length extending between about ½ to ⅔ of a circumference of said post.

17. The pen needle assembly of claim 14, wherein said spiral thread on said post has a pitch of about 1.0 to 1.5 times the longitudinal length of said post.

18. A method of assembling a pen needle assembly on a pen needle delivery device comprising the steps of:
coupling the pen needle assembly to the pen needle delivery device, where said pen needle assembly includes a needle hub with a post supporting a needle, an inner shield threaded to said post, and an outer shield covering said needle hub and inner shield; and
removing said outer shield to expose said inner shield, and rotating said inner shield relative to said post to disconnect said inner shield from said post and removing said inner shield to expose said needle.

19. The method of claim 18, wherein said outer surface of said post includes a spiral thread, and said threaded coupling mechanism of said inner shield is a spiral thread on an inner surface of said inner shield and complementing said spiral thread on said post.

20. The method of claim 19, wherein said spiral thread on said post extends between a proximal end of said post and said distal end of said post, and where said spiral thread has a pitch greater than or equal to a longitudinal length of said post.

21. The method of claim 19, wherein said spiral thread on said post has a pitch greater than a longitudinal length of said post.

22. The method of claim 19, wherein said spiral thread on said post has a length extending about ½ to ⅔ of a circumference of said post.

23. The method of claim 19, wherein said spiral thread on said post has a pitch of about 1.0 to 1.5 times the longitudinal dimension of said post.
